(19) United States
(12) Patent Application Publication
(10) Pub. No.: US 2024/0189367 A1
(43) Pub. Date: Jun. 13, 2024

(54) COMPOSITIONS FOR PROTECTION AGAINST PATHOGEN INFECTION AND METHOD THEREOF

(71) Applicant: HOLISTA BIOTECH SDN BHD, Petaling Jaya (MY)

(72) Inventors: M. Rajendran A/L V. MARNICKAVASAGAR, Petaling Jaya (MY); Roscoe Michael MOORE, JR., Rockville, MD (US); Arthur Victor MARTIN, Bluffton, SC (US)

(73) Assignee: HOLISTA BIOTECH SDN BHD, Petaling Jaya (MY)

(21) Appl. No.: 18/444,429

(22) Filed: Feb. 16, 2024

Related U.S. Application Data
(62) Division of application No. 17/716,892, filed on Apr. 8, 2022.

(51) Int. Cl. A61K 35/644; A61K 9/00; A61K 31/722; A61K 33/30; A61K 36/63; A61K 36/886; A61K 45/06

(52) U.S. Cl. CPC A61K 35/644; A61K 9/0043; A61K 31/722; A61K 33/30; A61K 36/63; A61K 36/886; A61K 45/06

(57) ABSTRACT

There is disclosed a composition for protection against pathogens, the composition comprising: predetermined amount of at least one type of plant-derived oil; predetermined amount of extracts from shea tree; predetermined amount of extracts from aloe vera; predetermined amount of wax; predetermined amount of chitosan; wherein the composition is in a form suitable to be applied to intranasal cavity. A method is disclosed herein.

### FIELD OF INVENTION

The present invention relates to preventing infections, and more particularly to a composition useful for protection against harmful pathogens, and method for preparing said composition.

### BACKGROUND

Microbiological pathogens are known as the primary cause of infectious diseases in humans or animals. The harmful effects of infections vary, in which they may differ from one organism to another. For humans and animals, the destructive effects including 15 horrific chronic diseases and, in some cases, leads to death. Pathogens have been one of the key contributions to deaths whereby the statistics reflecting the same have been tremendously growing over the past few years.

A great majority of reported infectious diseases can be spread directly, or indirectly between humans or between human and animals. Having said this, one of the many ways to effectively resolve this glaring problem is better control of infection, such as protecting exposed surfaces or preventing entry of the pathogens into the human body. One of the most common entry points of pathogens is the nasal cavity of both humans and animals. It is also known as one of the most vulnerably portions of the face that allows easy access to pathogens.

The nasal cavity is dark, damp and warm. The lining of the membranes is full of mucous secretions that are rich in both fatty and amino acids on a moist medium. This is classic environment for growth of pathogens once they are introduced into this space.

It would be a good idea to sanitize the nose just like you sanitize you hands and personal space. To do this, a broad spectrum anti-microbial is needed that: is effective, non toxic, non-irritant, has low dose, no odour, washes easily and does not interfere with the functioning of the structures of the nose.

Solutions involving prevention and their efficacy/effectiveness is verily dependent on the medicaments or compositions used, parameters/conditions and type of diseases.

Following the above, there is a need 5 to identify a solution to address at least one of the glaring issues with respect to prevention and protection against pathogens.

### SUMMARY

In one aspect, the invention provides a composition protection against pathogens, the composition comprising: predetermined amount of at least one type of plant-derived oil; predetermined amount of extracts from shea tree; predetermined amount of extracts from aloe vera; predetermined amount of wax; predetermined amount of chitosan; wherein the composition is in a form suitable to be applied to intranasal cavity.

Preferably, the composition further comprises zinc oxide.

Preferably, the plant derived oil includes at least one of the following: castor seed oil, evening primrose, jojoba, sweet almond; pumpkin seed oil or a combination thereof.

Preferably, the composition further comprises ascorbic acid, glycerol, citrus extracts, and plant-derived sugar.

Preferably, the composition further comprises at least one type of organosilicon compound.

Preferably, the composition controls infections by pathogens.

Preferably, the composition is applied as a lining within the nasal cavity of a patient.

Preferably, the extracts from shea tree includes shea butter.

Preferably, the organosilicon compound is polysilanes.

Preferably, the pathogens include harmful viruses, bacteria and fungi.

Preferably, the composition is prepared in nasal balm form.

Preferably, the organosilicon compound prolongs the efficacy of the composition upon contact with skin.

Preferably, the composition further includes edible oil.

In a further aspect, the present invention provides a method of preparing an intranasal cavity composition suitable for providing protection from pathogens, the method comprising:
a) preparing a mixture containing a predetermined amount of fruit or part of a fruit infused with herbs; a predetermined amount of extracts from shea tree; a predetermined amount of wax; a predetermined amount of chitosan; and predetermined amount of at least one type of organosilicon compound;
b) melting all ingredients from a) in a double boiler or small glass bowl over a small saucepan of water;
c) allow the mixture to simmer for a predetermined period until the oil has turned into a different color;
d) stirring well and pour into individual containers;
e) leaving the mixture in room temperature for a period of time for storage.

Preferably, the mixture is simmered for 3 to 4 hours under low heat.

Preferably, the method further includes adding oil into the mixture.

Preferably, the fruit is olive.

### DETAILED DESCRIPTION OF THE PREFERRED EMBODIMENT(S)

Further understanding of the object, construction, characteristics and functions of the invention, a detailed description with reference to the embodiments is given in the following.

Embodiments of the present invention are directed to a composition suitable for protection from pathogens, and method of preparation thereof. In one embodiment, the composition is prepared in a form suitable for intranasal cavity application.

It should be noted that the term “nasal cavity,” refers to all aspects of the nasal cavity, including the nostrils, nasal/mucus membrane, cilia, and sinuses, while the term “intranasal space” refers to the special cavity inside the nose.

The composition in accordance with the preferred embodiments of the present invention comprises natural ingredients, hence safe and environmentally friendly, with excipients that are easy to wash or removed from skin.

A broad spectrum anti-pathogenic solution based on citrus bioflavonoids that is effective is small concentrations, non-irritant, stable to heat, stable to light and stable over time.

The composition of the present invention provides protection against harmful viruses, bacteria and fungi, whereby it provides prolonged efficacy against the pathogens, compared to conventional compositions; when applied as a lining on the nasal membranes. The applied layer of the composition therefore traps and kill air borne pathogens that may have entered the nasal cavity. In one embodiment, the composition provides a non-irritant, colorless, odorless and non-staining medicament or agent for prevention of pathogen infections.

Accordingly, the composition sanitizes the nose without disrupting the nasal flora.

In a first aspect, the composition in accordance with a preferred embodiment of the present invention comprises: predetermined amount of olive(s) infused with herbs; predetermined amount of extracts from shea tree; predetermined amount of wax; predetermined amount of chitosan; and predetermined amount of at least one type of organosilicon compound.

In a second aspect, the composition further comprises at least one inorganic compound for example, zinc oxide, citrus extracts, glycerol, plant-derived sugar, and vitamins.

In accordance with a preferred embodiment of the present invention, the composition comprises castor seed oil; white beeswax sweet almond oil; evening primrose oil; aloe butter; shea butter; jojoba oil; Zinc Oxide; Vitamin E, glycerol, citrus extracts, plant-derived sugar, ascorbic acid, chitosan and pumpkin seed oil. These ingredients are provided in predetermined amounts in weight percentage.

Extracts from shea tree includes butter-based form extracted from the fruit kernels of shea tree, or any part of the plant. The preparation or extraction method may be of conventional processes, in which generally, the fruit is picked, cracked, roasted and pounded to extract the butter. The process may not or may include chemicals or synthetic agents to provide a suitable and safe form for application 5 on human skin. The shea butter in accordance with the preferred embodiment of the present invention provides soothing and protecting to skin with vitamins and healthy acids.

The herbs or plants in accordance with a preferred embodiment of the present invention may be of any type of herbs known for having homeopathic, medicinal, pharmaceutical properties, such as, but not limiting to; citrus based plants flaxseed, gingko, turmeric, evening primrose, Melaleuca alternifolia, echinacea, grapeseed, lavender, basil, catnip, cayenne, aloe vera, lavender, garlic, parsley, marigold, peppermint, rosemary, sage, thyme and chamomile.

In accordance with a preferred embodiment of the present invention, olives may be substituted with other suitable types of fruits or plants that can be obtained naturally. The olive or other type of plants may be prepared in the form of oil or extracts suitable for infusion with herbs as discussed above. The herbs may be infused by conventional means, processes or methods, such as soaking the fruit or part of fruit into a mixture of herbs with or without applying high temperature to the mixture.

Polysilanes in accordance with a preferred embodiment of the present invention may be substituted with other silicon-containing/organosilicon compounds, such as, but not limiting to polysiloxanes. It is known that these compounds exhibit good thermal stability, low viscosity and good gas permeability. These compounds may be prepared or synthesize by means of conventional chemical reactions. Polysilanes facilitate in providing a long lasting or prolongs the protective properties of the composition upon applied within the cavity.

A predetermined amount of wax or any similar organic compounds that can provide protective properties against skin damage is added to the composition to enable the preparation of the composition in balm form.

In a preferred embodiment, the composition of the present invention further comprises a predetermined amount of chitosan, which is unique polymer matrix that effectively traps pathogen specifically coronatype viruses.

In another preferred embodiment, the composition of the present invention may include ascorbic acid, vitamins and plant-derived sugar.

In a further aspect, the present invention provides a method for preparing a composition for protection against harmful pathogens. In the preferred embodiment, the composition is prepared as a nasal balm.

An example of the content of the composition in accordance with a preferred embodiment of the present invention (in weight percentage): 49% by weight of castor seed oil: 9% by weight of white beeswax; 6% by weight of sweet almond oil; 6% by weight of evening primrose oil; 0.5% by weight of aloe butter; 3.0% by weight of shea butter; 0.75% by weight of jojoba oil; 24% by weight of Zinc Oxide; 1.0% by weight of Vitamin E, 0.75% by weight of pumpkin seed oil; 3% by weight of a combination of citrus extracts,

Accordingly, the method of preparing an intranasal cavity composition suitable for providing protection from pathogens in accordance with a preferred embodiment of the present invention comprises the steps of:
f) preparing a mixture containing a predetermined amount of a fruit or part of a fruit infused with herbs; a predetermined amount of extracts from shea tree; a predetermined amount of wax; a predetermined amount of chitosan; and predetermined amount of at least one type of organosilicon compound;
g) melting all ingredients from a) in a double boiler or small glass bowl over a small saucepan of water;
h) allow the mixture to simmer for a predetermined period until the oil has turned into a different color;
i) stirring well and pour into individual containers;
j) leaving the mixture in room temperature for a period of time for storage.

The simmering is conducted for an estimated period of 3 to 4 hours under low heat, preferably in a controlled manner so as not to destroy the properties of the mixture. It is anticipated that the mixture may further include at least one type of edible oil, preferably vegetable oil to provide nutritional richness. In a preferred 5 embodiment, the edible oil is one of the following groups of edible oils: palm oil, coconut oil, canola oil, soybean oil, olive oil, jojoba oil, castor oil, pumpkin seed oil, and rapeseed oil and/or a combination thereof.

In the preferred embodiment, to use the composition or nasal balm, the user applies a small amount of the balm to the affected area, particularly on the skin surface in the nasal cavity. The application is repeated until redness and soreness has subsided.

It is concluded that the composition provides anti-pathogenic effects against infections by microorganisms once applied on the nasal cavity of a patient. The nasal balm in accordance with the present invention therefore prevents the entry of pathogens into the human respiratory system. Understandably, the composition of the present invention may be used to inhibit or prevent further infection of harmful pathogens, including various types of coronaviruses, thereby prevent the spread of diseases due to these infectious pathogens.

Although the present invention has been described with reference to the preferred embodiments and examples thereof, it is apparent to those skilled in the art that a variety of modifications and changes may be made without departing from the scope of the present invention which is intended to be defined by the appended claims.

1. A method of preparing an intranasal cavity composition suitable for providing protection from pathogens, the method comprising:
a) preparing a mixture containing a predetermined amount of at least one type of plant-derived oil or a predetermined amount of fruit or part of a fruit infused with herbs; a predetermined amount of extracts from shea tree;
predetermined amount of extracts from aloe vera, a predetermined amount of wax; a predetermined amount of chitosan; at least an inorganic compound and predetermined amount of at least one type of organosilicon compound;
b) melting all ingredients from mixture of step a);
c) allow the mixture to simmer for a predetermined period until the oil has turned into a different color;
d) stirring well and pour into individual containers;
e) leaving the mixture in room temperature for a period of time for storage.

2. The method as claimed in claim 1, wherein the mixture is simmered for 3 to 4 hours under low heat.

3. The method as claimed in claim 1, wherein the method further includes adding oil into the mixture selected from palm oil, coconut oil, canola oil, soybean oil, olive oil, and rapeseed oil and/or a combination thereof.

4. The method as claimed in claim 1, wherein the fruit is olive.

5. The method as claimed in claim 1, wherein the organosilicon compound is polysilanes.

6. The method as claimed in claim 1, wherein the method further comprises herbs or plants having homeopathic, medicinal, pharmaceutical properties selected from the group comprising citrus-based plants flaxseed, gingko, turmeric, evening primrose, Melaleuca alternifolia, echinacea, grapeseed, lavender, basil, catnip, cayenne, aloe vera, lavender, garlic, parsley, marigold, peppermint, rosemary, sage, thyme and chamomile.

7. A method of preparing an intranasal cavity composition suitable for providing protection from pathogens comprising
i) mixing about 49% by weight of castor seed oil, about 9% by weight of white beeswax;
about 6% by weight of sweet almond oil; about 6% by weight of evening primrose oil;
about 0.5% by weight of aloe butter; about 3.0% by weight of shea butter; about 0.75% by weight of jojoba oil; about 24% by weight of Zinc Oxide; about 1.0% by weight of Vitamin E, about 0.75% by weight of pumpkin seed oil; about 3% by weight of a combination of citrus extracts, glycerol, ascorbic acid, sugar and chitosan;
ii) melting all ingredients of mixture from step i);
ii) allowing the obtained mixture to simmer for 3-4 hours until the oil has turned into a different color;
iii) stirring well the mixture obtained in step ii) and pouring into individual containers;
iv) leaving the mixture in room temperature for a period of time for storage.
